(19) United States
(12) Patent Application Publication
(10) Pub. No.: US 2025/0356278 A1
(43) Pub. Date: Nov. 20, 2025

(54) MATCHING SERVER, MATCING SYSTEM, AND MATHICNG METHOD

(71) Applicant: OLYMPUS CORPORATION, Tokyo (JP)

(72) Inventors: Arata SHINOZAKI, Tokyo (JP); Yuta OKADA, Tokyo (JP); Yoshiro OKAZAKI, Tokyo (JP); Yuji SAKAMOTO, Tokyo (JP); Tatsuki KUNO, Tokyo (JP)

(73) Assignee: OLYMPUS CORPORATION, Tokyo (JP)

(21) Appl. No.: 19/284,919

(22) Filed: Jul. 30, 2025

Related U.S. Application Data
(63) Continuation of application No. PCT/JP2023/003653, filed on Feb. 3, 2023.

(51) Int. Cl. G06Q 10/0631; G06Q 30/0283; G16H 40/20

(52) U.S. Cl. CPC G06Q 10/063112; G06Q 30/0283; G16H 40/20

(57) ABSTRACT

A matching server includes a processor configured to: in matching a user who requests support work and a supporter who provides the support work to the user to receive compensation from the user, calculate a setting range of the compensation that the supporter is able to set; and notify the supporter of the setting range.

### CROSS-REFERENCE TO RELATED APPLICATION

This application is a continuation of International Application No. PCT/JP2023/003653, filed on Feb. 3, 2023, the entire contents of which are incorporated herein by reference.

### BACKGROUND

### 1. Technical Field

The present disclosure relates to a matching server, a matching system, and a matching method.

### 2. Related Art

In a matching system, a technique of presenting a candidate instantaneously to an organization that requests recruiting temporarily has been known (for example, refer to Japanese Patent No. 6972257). In the technique, classification is performed based on a given condition on a candidate information storage unit that stores information on candidates who want to work and conditions and, after recruiting conditions of the organization that requests recruiting and information on the candidates on which classification is performed are referred to and information on a candidate that matches the recruiting conditions is displayed, the candidate that matches the recruiting conditions is informed.

### SUMMARY

In some embodiments, a matching server includes a processor configured to: in matching a user who requests support work and a supporter who provides the support work to the user to receive compensation from the user, calculate a setting range of the compensation that the supporter is able to set; and notify the supporter of the setting range.

In some embodiments, A matching system includes: a matching server including a processor; and a supporter terminal device that is associated with a supporter who provides support work to a user to receive compensation from the user. The processor is configured to in matching the user who requests the support work and the supporter, calculate a setting range of the compensation that the supporter is able to set, and output the setting range to the supporter terminal device.

In some embodiments, provided is a matching method executed by a matching server including a processor. The processor is configured to execute, in matching a user who requests support work and a supporter who provides the support work to the user to receive compensation from the user, calculating a setting range of the compensation that the supporter is able to set, and notifying the supporter of the setting range.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a matching system according to a first embodiment;

FIG. 2 is a schematic diagram illustrating a schematic configuration of a front side of medical equipment according to the first embodiment;

FIG. 3 is a schematic diagram illustrating a schematic configuration of a back side of the medical equipment according to the first embodiment;

FIG. 4 is a block diagram illustrating a functional configuration of the medical equipment according to the first embodiment;

FIG. 5 is a block diagram illustrating a functional configuration of a matching server according to the first embodiment;

FIG. 6 is a diagram illustrating an overview of a method of setting compensation for supporter by a matching server according to the first embodiment;

FIG. 7 is a diagram illustrating an overview of a method of matching a user and a supporter performed by the matching server according to the first embodiment;

FIG. 8 is a flowchart illustrating an overview of a process that the matching server executes according to the first embodiment;

FIG. 9 is a block diagram illustrating a functional configuration of a matching server according to a second embodiment;

FIG. 10 is a flowchart illustrating an overview of a process that the matching server executes according to the second embodiment;

FIG. 11 is a block diagram illustrating a functional configuration of a matching server according to a third embodiment;

FIG. 12 is a flowchart illustrating an overview of a process that a matching server executes according to a fourth embodiment;

FIG. 13 is a block diagram illustrating a functional configuration of the matching server according to the fourth embodiment; and

FIG. 14 is a flowchart illustrating an overview of a process that the matching server executes according to the fourth embodiment.

### DETAILED DESCRIPTION

Modes for carrying out the disclosure will be described in detail below with the drawings in respect of a matching system including a matching server. Note that the following embodiments do not limit the disclosure. In the following description, each of the drawings referred to only schematically illustrates the shapes, the sizes, and the positional relationships such that the content of the disclosure is understandable. In other words, the disclosure is not limited to only the shapes, the sizes, and the positional relationships that are exemplified in the respective drawings.

### First Embodiment

### Schematic Configuration of Matching System

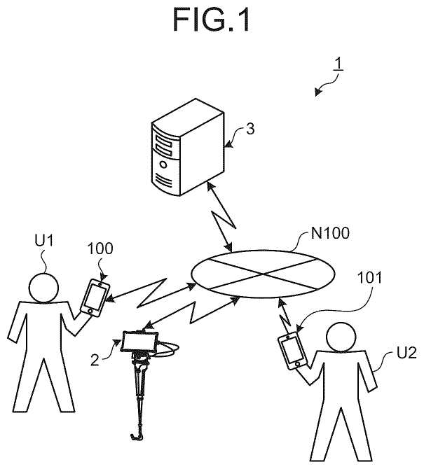

FIG. 1 is a schematic diagram illustrating a schematic configuration of a matching system according to a first embodiment. A matching system 1 illustrated in FIG. 1 is one for matching with a supporter U2 who provides support work, such as teaching, coaching, a lecture and a course, for performing a method of using medical equipment and medical procedure, a treatment method, an examination method, a treatment method, and a diagnosis method using the medical equipment (simply referred to as “medical method” below) that a user U1 requests via a network N100.

The user U1 here is a healthcare professional, such as a doctor who makes a medical practice, and is an unskilled person in the requested medical method. The supporter U2 is a healthcare professional, such as a doctor who makes a medical practice, and is a skilled person in the medical method that the user U2 requests. Furthermore, the skilled person has passed through a given training time in the given medical method.

As illustrated in FIG. 1, the matching system 1 includes medical equipment 2 that the user U1 or the supporter U2 who is a doctor in a medical institution, or the like, uses in medical care, such as an examination, a diagnosis, and a treatment, and a matching server 3 capable of communicating with the medical equipment 2, a user terminal device 100, and a supporter terminal device 101 via the network N100. A network consisting of the Internet and a mobile phone network, or the like, is assumed as the network N100. Note that the medical equipment 2 and the user terminal device 100 are independent in FIG. 1; however, they are not limited to this and they may be a network connection terminal device or medical equipment in which functions of the medical equipment 2 and the user terminal device 100 are formed integrally or the network connection terminal device may be detachable from the medical equipment 2.

Note that FIG. 1 illustrates the case where each of the user U1 and the supporter U2 is single to simplify the following description; however, they are not limited to this and there are a plurality of the users U1 and a plurality of the supporters U2. In other words, each of the users U1 accesses the matching server 3 via the network N100 using the user terminal device 100 of the user U1 and selects the supporter U2 who provides support work for the requested medical method from among the supporters U2 who are registered in the matching server 3 previously to receive the requested support work.

### User Terminal Device and Supporter Terminal Device

First of all, the user terminal device 100 and the supporter terminal device 101 will be described.

The user terminal device 100 and the supporter terminal device 101 are configured using a mobile phone, a PC, such as a personal computer, a tablet terminal device, or the like, that includes at least an input interface, such as a touch panel, a communication function enabling connection to the network N100, and a display monitor capable of displaying information, such as an image.

### Configuration of Medical Equipment

The medical equipment 2 will be described next.

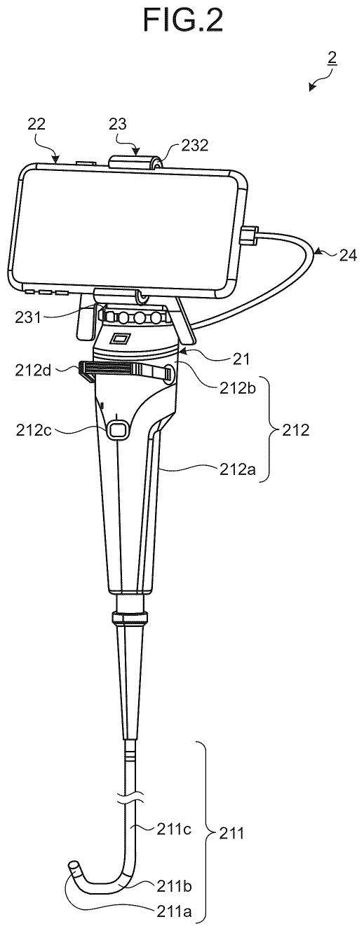

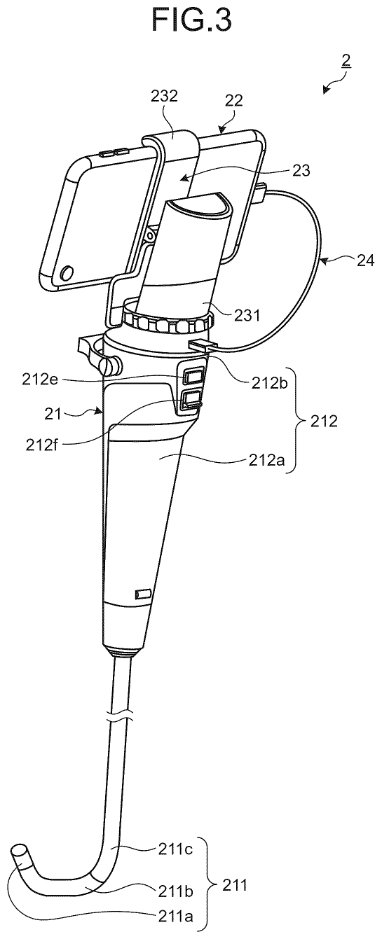

FIG. 2 is a schematic diagram illustrating a schematic configuration of a front side of the medical equipment 2. FIG. 3 is a schematic diagram illustrating a schematic configuration of a back side of the medical equipment 2.

The medical equipment 2 illustrated in FIG. 2 and FIG. 3 is an endoscope that is used in the field of medicine, that is inserted into a subject, such as a human or an animal, and that displays a live view image corresponding to data of captured internal images of the subject, thereby observing or treating the subject. A soft upper gastrointestinal general-purpose endoscope will be exemplified and described below as the medical equipment 2 and the endoscope is required to be capable of communicating with the user terminal device 100, the supporter terminal device 101, and the matching server 3. For example, the medical equipment may be a MRI (Magnetic Resonance Imaging), a CT (Computed Tomography), an ultrasound endoscope, an abdominal ultrasound examination apparatus, a high-frequency knife, a high-frequency ablation power device, a medical video system, a surgical energy device, or a laparoscope system. Needless to say, an accessory, such as a communication device with a communication function may be additionally detachable from the medical equipment 2 and the medical equipment 2 may be connected to the network N100 via the accessory.

Furthermore, the medical equipment 2 may be connected to the network N100 via the user terminal device 100 or the supporter terminal device 101. The user terminal device 100 and the network connection terminal device assumed to be used in the medical equipment 2 may be the same, the user terminal device 100 may be connected to the medical equipment 2 and used, or the network terminal device may be detached from the medical equipment 2 and used as the user terminal device as in FIG. 2 described above.

As illustrated in FIG. 2 and FIG. 3, the medical equipment 2 includes a main unit 21, a display unit 22, a holder 23, and a communication cable 24.

First of all, the main unit 21 will be described.

The main unit 21 is inserted into a subject, captures an internal image of the subject, and generates data of the captured internal image of the subject. The main unit 21 includes an insertion unit 211 that is inserted into the subject and an operation unit 212 that is on a side of a proximal-end part of the insertion unit 211 and that a practitioner, such as a doctor, grips.

The insertion unit 211 incorporates an electronic cable, an operation wire, and the like. The insertion unit 211 includes an distal end part 211a having an illuminating unit incorporating a white LED (Light Emitting Diode) lamp that emits illuminating light to the subject, an illuminating lens, and the like, and an imaging unit incorporating an imaging device that captures an internal image of the subject and, an imaging lens, and the like; a curve part 211b that consists of a plurality of curve pieces and that flexibly curves; and a flexible tube 211c that is provided on a side of a proximal end part of the curve part 211b.

The operation unit 212 includes a grip 212a to which a proximal end part of the flexible tube 211c is connected and that enables the practitioner to grip the main unit 21 and a housing 212b that is provided on a side of a proximal end part of the grip 212a. The housing 212b has various types of control circuits, batteries, and the like, inside. The housing 212b has a start button 212c that starts the power of the main unit 21, a curve operation lever 212d that causes the curve part 211b to curve in up and down directions; a first button 212e that receives an input of an instruction signal that provides an instruction to capture an image; and a second button 212f to which a given function can be assigned.

The display unit 22 will be described next.

The display unit 22 displays a display image corresponding to the data on the captured internal image of the subject obtained by capturing an internal image of the subject, which is data input from the main unit 21, and various types of information on the medical equipment 2. The display unit 22 is configured using a memory, a processor including hardware, such as a CPU (Central Processing Unit), a FPGA, or a ASIC (Application Specific Integrated Circuit), a display monitor of a liquid crystal display or an organic electroluminescent display, etc. The display unit 22 performs various types of processing on various types of data that are input from the imaging unit of the distal end part 211a of the main unit 21 via the communication cable 24 and makes a display. Note that a display area of the display unit 22 may be provided with a touch panel that receives inputs of various types of operations made by the user U1 and outputs signals corresponding to the content of the received operations to the main unit 21 via the communication cable 24, or the like. The display unit 22 may consist of a mobile phone, a smartphone, a tablet terminal device, or the like, that is detachable from the main unit 21.

The holder 23 will be described next.

The holder 23 is detachably attached to the medical equipment 2 and holds the display unit 22. The holder 23 includes a holder supporting unit 231 that extends from the operation unit 212 of the main unit 21 and is formed in an integrated manner and a holding unit 232 that is detachable from the holder supporting unit 231 and that holds the display unit 22 detachably. The holding unit 232 enables holding in a longitudinal direction and a lateral direction of the display unit 22. The state where the holder 23 holds the display unit 22 in the longitudinal direction will be described below.

The communication cable 24 will be described next.

The communication cable 24 connects the main unit 21 and the display unit 22 such that they are able to communicate with each other. Specifically, the communication cable 24 outputs various types of data that are input from an imaging camera that is incorporated in the distal end part 211a of the main unit 21 to the display unit 22. The data that is input to the display unit 22 is processed by a processor in the display unit 22, or the like. Note that, in the first embodiment, the main unit 21 and the display unit 22 are connected such that they are able to communicate with each other; however, they are not limited to this and the main unit 21 and the display unit 22 may be connected using a given radio communication standard, such as Wi-Fi (trademark) and Bluetooth (trademark), such that they are able to communicate with each other.

### Functional Communication of Medical Equipment

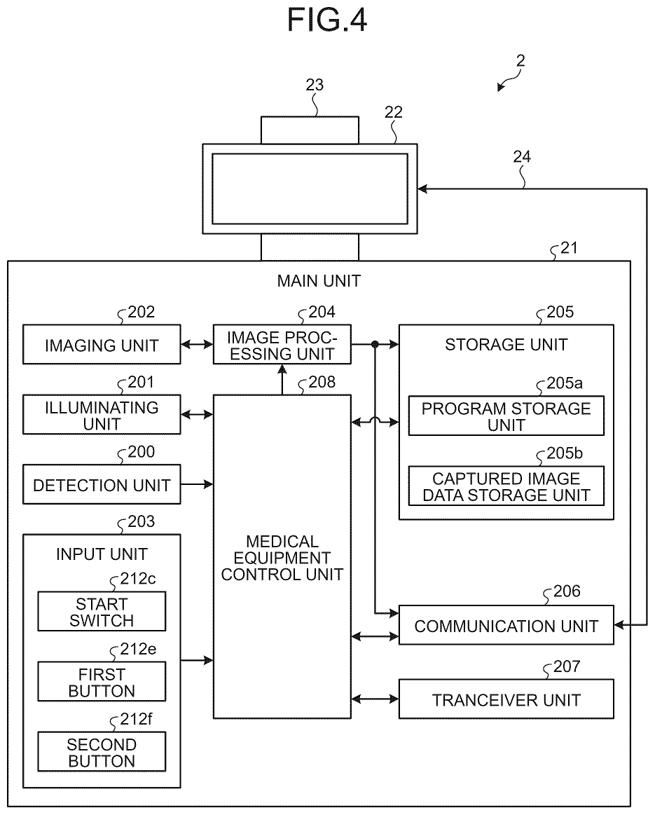

A functional Configuration of the medical equipment will be described next. FIG. 4 is a block diagram illustrating the functional configuration of the medical equipment 2.

As illustrated in FIG. 4, the medical equipment 2 includes a detection unit 200, an illuminating unit 201, an imaging unit 202, an input unit 203, an image processing unit 204, a storage unit 205, a communication unit 206, a transceiver unit 207, and a medical equipment control unit 208.

The detection unit 200 detects a posture and a position of the insertion unit 211 and outputs the result of the detection to the medical equipment control unit 208. The detection unit 200 is configured using a gyro sensor, an acceleration sensor, and the like.

The illuminating unit 201 emits illuminating light to the subject under the control of the medical equipment control unit 208. The illuminating unit 201 is configured using a white LED lamp capable of emitting white illuminating light and an illuminating lens that concentrates the illuminating light that is emitted by the white LED lamp and emits the concentrated illuminating light to the subject.

The imaging unit 202 generates captured image data by capturing an image of the subject to which the illuminating unit 201 illuminates light and outputs the captured image data to the image processing unit 204. The imaging unit 202 is configured using one or a plurality of imaging lenses that concentrate light from a given view area and an image sensor, such as a CCD (Charge Coupled Device) image sensor that receives a subject image that is formed by the imaging lens and that generates captured image data by performing photoelectric conversion or a CMOS (Complementary Metal Oxide Semiconductor) image sensor.

The input unit 203 includes a start button 212c that starts the power of the medical equipment 2, a first button 212e that receives an input of an imaging operation and that outputs an imaging signal corresponding to the received imaging operation to the medical equipment control unit 208, and a second button 212f to which a given function can be assigned.

Under the control of the medical equipment control unit 208, the image processing unit 204 performs given image processing on the captured image data that is input from the imaging unit 202 and outputs the captured image data to at least any one of the storage unit 205 and the communication unit 206. The given image processing here is image processing including at least A/D conversion processing, gain adjustment processing, optical black subtraction processing, and white balance (WB) adjustment processing, and, in the case where the imaging device has a Bayer arrangement, concurrent processing, color matrix operation processing, gamma correction processing, color reproduction processing, and edge enhancement processing. The image processing unit 204 is configured using a processor including a memory, and hardware, such as a FPGA (Field Programmable Gate Array, an ASIC (Application Specific Integrated Circuit), and a GPU (Graphics Processing Unit).

The storage unit 205 is realized using a ROM (Read Only Memory), RAM (Random Access Memory), a flash memory, etc. The storage unit 205 includes a program storage unit 205a that records various types of programs that the medical equipment 2 executes and various types of data used during processing and a captured image data storage unit 205b that records the captured image data. Note that the storage unit 205 may be a recording medium, such as a memory card that is detachable from the medical equipment 2.

The communication cable 24 is detachably connected to the communication unit 206 and the communication unit 206 performs communication with the display unit 22 under the control of the medical equipment control unit 208. For example, the communication unit 206 transmits the captured image data to the display unit 22 according to a communication standard, such as HDMI (High-Definition Multimedia Interface) (trademark). The communication unit 206 is configured using a male connector according to the USB-IF standard, or the like.

Under the control of the medical equipment control unit 208, the transceiver unit 207 transmits various types of information to the matching server 3, the user terminal device 100, or the supporter terminal device 101 and receives various types of information from the matching server 3, the user terminal device 100, or the supporter terminal device 101 via the network N100. The transceiver unit 207 is configured using a communication module capable of transmitting and receiving various types of information, or the like.

The medical equipment control unit 208 is configured using a processor including a memory and hardware, such as a CPU (Central Processing Unit), a FPGA, or an ASIC (Application Specific Integrated Circuit). The medical equipment control unit 208 controls each unit forming the medical equipment 2. Note that, in the first embodiment, the image processing unit 204, a storage unit 25, and the medical equipment control unit 208 are provided in the main unit 21; however, they are not limited to this and, for example, part or all of the image processing unit 204, the storage unit 25, and the medical equipment control unit 208 may be provided in the display unit 22.

### Functional Configuration of Matching Server

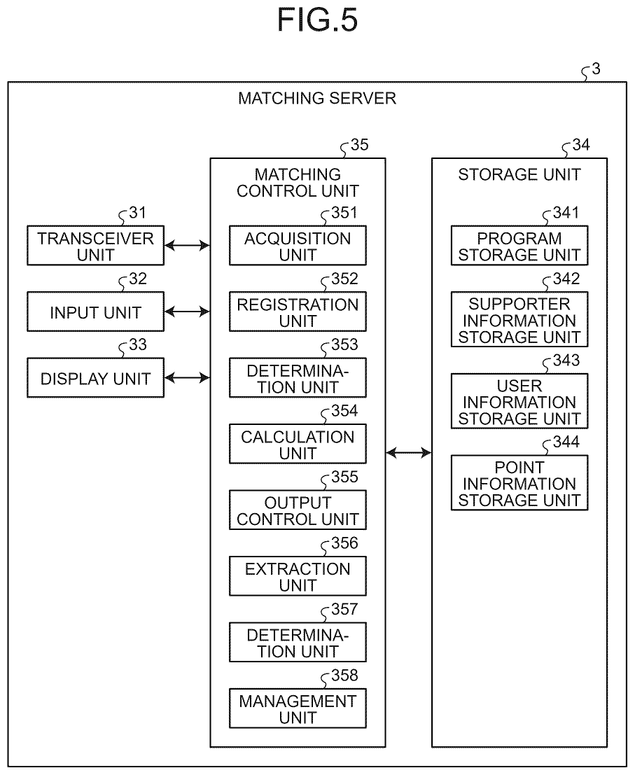

A functional configuration of the matching server 3 will be described next. FIG. 5 is a block diagram illustrating a functional configuration of the matching server 3. The matching server 3 stores supporter information on a plurality of the supporters U2, extracts the supporter U2 who is able to provide support work of a medical method that the user U1 requests according to an access of the user terminal device 100 that the user U1 owns, and matches the supporter U2 with the user.

The matching server 3 includes a transceiver unit 31, an input unit 32, a display unit 33, a storage unit 34, and a matching control unit 35.

Under the control of the matching control unit 35, the transceiver unit 31 transmits various types of information to the medical equipment 2, the user terminal device 100, and the supporter terminal device 101 and receives various types of information from the medical equipment 2, the user terminal device 100, and the supporter terminal device 101 via the network N100. The transceiver unit 31 is configured using a communication module capable of transmitting and receiving various types of information, or the like.

The input unit 32 is configured using a keyboard, a mouse, a switch, a touch panel, etc., receives inputs of various types of operations of the user, and outputs information corresponding to the received operations to the matching control unit 35.

Under the control of the matching control unit 35, the display unit 33 displays various types of information on the matching server 3. The display unit 33 is configured using a displaying display, such as a liquid crystal display or an organic EL display.

The storage unit 34 is configured using a DRAM, a ROM, a flash memory, a HDD (Hard Disk Drive), a SSD (Solid State Drive), etc. The storage unit 34 records various types of information on the matching server 3. The storage unit 34 includes a program storage unit 341 that records various types of programs that the matching server 3 executes, a supporter information storage unit 342 that stores supporter information on each of the supporters, a user information storage unit 343 that stores user information on each of the users, and a point information storage unit 344 that stores point information on points that each of the supporters and each of the users is able to use. The supporter information here contains a supporter ID that identifies the supporter U2, medical procedure and medical equipment that the supporter U2 is able to teach in support work, the number of times of experience, the number of times of performance, the number of times support work is provided, the number of times the supporter U2 taught support work to other users in the past, a provision time and date and time for which and at which the supporter U2 is able to provide support work, the number of years after acquisition of a medical license, an address, contact information, language, and a time during which contact can be made. The user information contains a user ID that identifies the user U1, the number of years after acquisition of a medical license, an address, a level of proficiency in each set of medical procedure, the number of times the user U1 is taught support work in the past, a supporter ID of the supporter U2 that teaches support work. The point information is on points usable in a given payment method (for example, a two-dimensional code payment method) by which the user U1 or the supporter U2 make a purchase using his/her credit card, legal tender, virtual currency, electronic money, or the like. Needless to say, the point information may be associated with a credit card number, a bank account, an electronic payment service number that is associated with each of the user U1 and the supporter U2.

Back to FIG. 5, description of the matching server 3 will be continued.

The matching control unit 35 is configured using a memory and a processor including hardware, such as a CPU, a FPGA, or an ASIC. The matching control unit 35 controls each unit forming the matching server 3. The matching control unit 35 reads a program that is stored in the program storage unit 341 into a work area of the memory, executes the program, and controls each component, and the like, through execution of the program by the processor and accordingly the hardware and the software cooperate and realize a functional module that matches a given purpose. Note that, in the first embodiment, the matching control unit 35 functions as the processor. Specifically, in matching a user who requests support work of teaching a method of using medical equipment and medical procedure and a supporter who provides the support work to the user and obtains compensation from the user, the matching control unit 35 calculates a setting range representing a range of compensation that the supporter is able to set and notifies the supporter of the setting range.

The matching control unit 35 includes an acquisition unit 351, a registration unit 352, a determination unit 353, a calculation unit 354, an output control unit 355, an extraction unit 356, a determination unit 357, and a management unit 358.

The acquisition unit 351 acquires the supporter information on the supporter U2 that is input by the supporter U2 using the supporter terminal device 101 from the supporter terminal device 101. The acquisition unit 351 acquires compensation information on compensation for providing the support work that is selected by the supporter U2 from the setting range of compensation that is displayed on the supporter terminal device 101 from the supporter terminal device 101. The acquisition unit 351 acquires a matching condition and the user information that are input by the user U1 using the user terminal device 100. Based on the user information, the acquisition unit 351 acquires the point information that the user U1 has and that is associated with the user U1 from the point information storage unit 344.

The registration unit 352 registers the user information on the user U1 that is acquired by the acquisition unit 351 from the user terminal device 100 in the user information storage unit 343. The registration unit 352 registers the supporter information on the supporter U2 that is acquired by the acquisition unit 351 from the supporter terminal device 101 in the supporter information storage unit 342. Furthermore, the registration unit 352 stores the compensation information on the supporter U2 that is acquired by the acquisition unit 351 from the supporter terminal device 101 in the supporter information storage unit 342 in association with the supporter ID.

Based on the supporter information on the supporter U2, the determination unit 353 determines a rate of the supporter U2 serving as an index representing the quality of the support work.

Based on the rate that is determined by the determination unit 353, the calculation unit 354 calculates a range of setting compensation to be received from the user U1 for providing the support work that can be set by the supporter U2.

The output control unit 355 transmits the setting range of compensation that is calculated by the calculation unit 354 to the supporter terminal device 101 of the supporter U2.

Based on the point information that the user U1 has and the matching condition that is acquired from the user terminal device 100 of the user U1, the extraction unit 356 extracts the supporter U2 who is able to provide medical procedure that is within a range of points usable by the user U1 and that the user U1 requests from the supporter information storage unit 342.

The determination unit 357 determines whether approval information on approval of provision of the support work is received from the supporter terminal device 101 of the supporter U2.

The management unit 358 makes a payment of the compensation corresponding to the support work from the point information of the user U1. Specifically, based on the point information on the user U1 and the point information on the supporter U2 that the point information storage unit 344 stores, the management unit 358 subtracts points corresponding to the setting range of the support work that the supporter U2 provides from the point information on the user U1 and adds the points corresponding to the setting range according to provision of the support work to the point information on the supporter U2, thereby making a payment of compensation from the user U1 to the supporter U2.

Overview of Method of Setting Compensation to Supporter by Matching Server

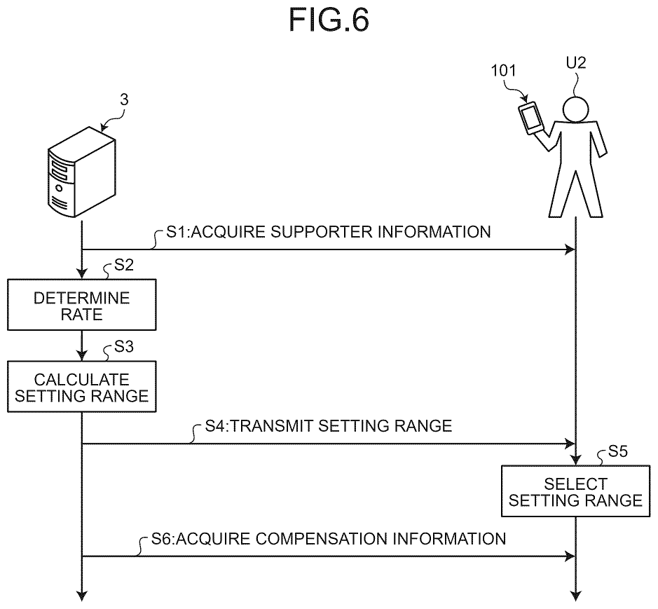

An overview of a method of setting compensation to the supporter U2 by the matching server 3 will be described next. FIG. 6 is a diagram illustrating the overview of the method of setting compensation to the supporter U2 by the matching server 3.

As illustrated in FIG. 6, the acquisition unit 351 acquires the supporter information on the supporter U2 that is input by the supporter U2 using the supporter terminal device 101 via the network N100 (step S1). The supporter information here contains the supporter ID that identifies the supporter U2, the medical procedure and medical equipment that the supporter U2 is able to teach in support work, the number of times of experience, the number of times of performance, the number of times support work is provided, the number of times the supporter U2 taught support work to other users in the past, the provision time and date and time for which and at which the supporter U2 is able to provide support work, the number of years after acquisition of a medical license, the address, the contact information, language, and the time during which contact can be made. In this case, the registration unit 352 stores the supporter information that is acquired by the acquisition unit 351 in the supporter information storage unit 342.

Subsequently, the determination unit 353 determines a rate of the supporter U2 (step S2). Specifically, the determination unit 353 determines, as the rate of the supporter U2, a value obtained by multiplying a level of medical procedure that can be taught in the support work that can be provided by the supporter U2, the level of proficiency of the medical procedure, a level of difficulty in dealing with the medical equipment 2 to be used, experiences, the number of times support work is provided, and the number of years after acquisition of the medical license that are contained in the supporter information. For example, the determination unit 353 determines that the rate of the supporter U2 is “3” based on the supporter information. Needless to say, the determination unit 353 determines a rate according to not only a numerical value simply but also another evaluation method. Furthermore, the determination unit 353 may determine, as a rate, a result of deep learning using a result of learning in which the level of medical procedure that can be taught, the level of proficiency of the medical procedure, the level of difficulty in dealing with the medical equipment 2 to be used, the experience, the number of times support work is provided, the number of years after acquisition of the medical license serves as input data, etc., serve as input data and the rate is output as output data by machine learning.

Thereafter, based on the rate of the supporter U2 that is determined by the determination unit 353, the calculation unit 354 calculates a range of setting compensation that can be received by the supporter U2 by providing the support work to the user U1 (step S3). For example, when the rate of the supporter U2 that is determined by the determination unit 353 is “3”, the calculation unit 354 calculates that the range of setting compensation is between $150 and $200 per hour. Note that the calculation unit 354 may calculate a range of setting compensation using a rate table in which a rate and a range of setting compensation that are previously set are associated with each other.

Subsequently, the output control unit 355 transmits the range of setting compensation that the supporter U2 is able to receive to the supporter terminal device 101 of the supporter U2 (step S4). Accordingly, the supporter U2 is able to know compensation for the support work that the supporter U2 is able to provide.

Thereafter, the supporter U2 selects requested compensation from the range of setting compensation that is transmitted from the matching server 3 and that is displayed on the supporter terminal device 101 (step S5).

Subsequently, the acquisition unit 351 acquires the compensation information on compensation necessary to receive the support work that is specified by the supporter U2 from the supporter terminal device 101 (step S6). In this case, the registration unit 352 stores the supporter information and the compensation information in association with each other in the supporter information storage unit 342. Furthermore, when the supporter U2 sets a requested value according to the setting range that is displayed by the supporter terminal device 101, the registration unit 352 determines compensation for the support work according to this setting operation and stores the determined compensation for the support work and the support work in association with each other in the supporter information storage unit 342.

As described above, according to the matching server 3, because the supporter U3 is not allowed to set exorbitant compensation freely by himself/herself and the supporter U2 is made set compensation within the range of setting compensation based on the rate of the supporter U2, it is possible to prevent compensation that does not match provided support work from being set.

Furthermore, because the range of setting compensation is not fixed values, the matching server 3 is able to set compensation corresponding to the quality of the support work. Note that, in the first embodiment, money will be described as an example of the compensation; however, the compensation is not limited thereto and electronic money, points usable in a specific service, or the like, may serve as the compensation.

Overview of Method of Matching User and Supporter by Matching Server

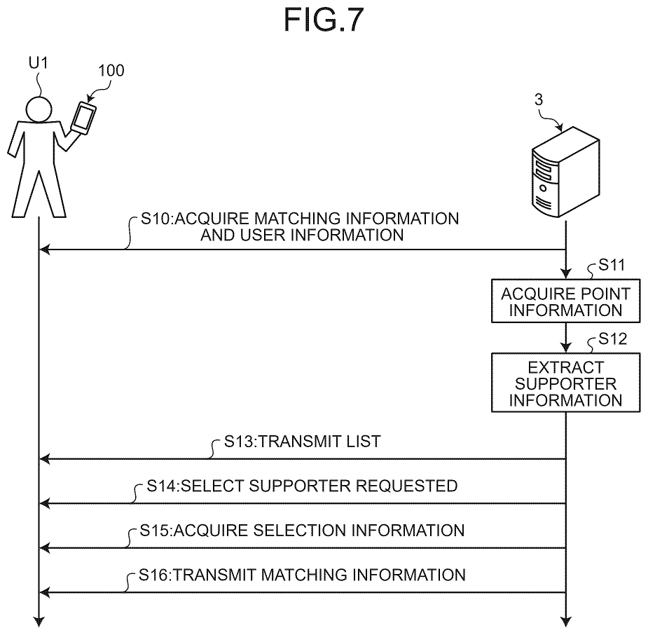

An overview of a method of matching the suer U1 and the supporter U2 by the matching server 3 will be described next. FIG. 7 is a diagram illustrating the overview of the method of matching the user U1 and the supporter U2 by the matching server 3.

As illustrated in FIG. 7, first of all, the acquisition unit 351 acquires a matching condition for matching with the supporter U2 and user information that are input by the user U1 to the user terminal device 100 (step S10). The matching condition contains at least a medical method using medical procedure that the user U1 requests and the medical equipment that is used for the medical method. The user information is, for example, the user ID that identifies the user U1, the level of proficiency in each set of medical procedure, experiences, the language, the address, etc. In this case, the registration unit 352 stores the user information that is acquired by the acquisition unit 351 in the user information storage unit 343.

Subsequently, the acquisition unit 351 acquires point information that is associated with the user ID of the user terminal device 100 of the user U1 (step S11). Specifically, the acquisition unit 351 acquires the point information associated with the user ID of the user terminal device 100 from the point information storage unit 344.

Thereafter, based on the point information that the user U1 has and the matching condition that is input by the user U1, the extraction unit 356 extracts one or more supporters U2 from which provision of the support work for the requested medical procedure or the medical equipment 2 can be received using the points that the user U1 has (step S12).

Subsequently, the output control unit 355 outputs a list of the one or more supporters U2 extracted by the extraction unit 356 to the user terminal device 100 (step S13).

Thereafter, the user U1 selects the supporter U2 whom the user U1 requests from the list of the supporters U2 that is transmitted from the matching server 3 and that is displayed on the user terminal device 100 (step S14).

Thereafter, the acquisition unit 351 acquires selection information on the selection of the requested supporter U2 from the list displayed on the user terminal device 100 by the user U1 (step S15).

Subsequently, based on the selection information that is acquired by the acquisition unit 351, the output control unit 355 transmits matching completion information indicating that matching is made to the user terminal device 100 of the user U1 and the supporter U2 that is selected by the user U1 (step S16).

As described above, the user U1 is able to receive provision of the support work on the requested medical procedure or the medical equipment 2 from the requested supporter U2 based on the points that the user U1 has.

Process performed by Matching Server

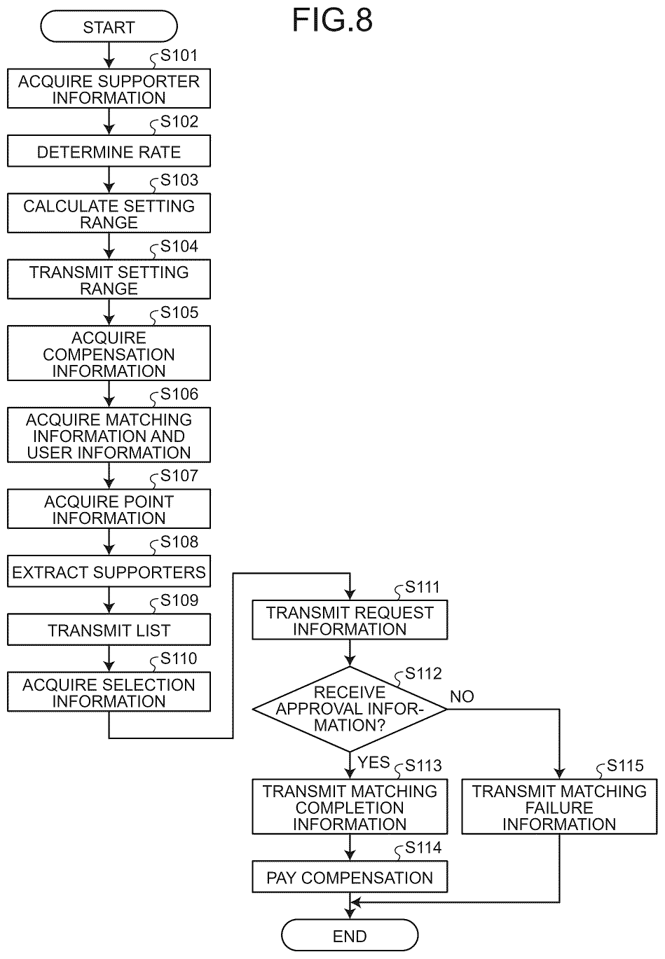

An overview of a process that the matching server 3 executes will be described next. FIG. 8 is a flowchart illustrating the overview of the process that the matching server 3 executes.

As illustrated in FIG. 8, first of all, the acquisition unit 351 acquires the supporter information on the supporter U2 that is input by the supporter U2 using the supporter terminal device 101 from the supporter terminal device 101 (step S101).

Subsequently, the determination unit 353 determines a rate of the supporter U2 based on the supporter information (step S102). In this case, the registration unit 352 stores the supporter information on the supporter U2 and the rate that is determined by the determination unit 353 in association with each other in the supporter information storage unit 342. Note that the determination unit 353 may determine and update a rate of the supporter U2 each time the acquisition unit 351 acquires supporter information from the supporter U2 in every give cycle, for example, biannually.

Subsequently, based on the rate hat is determined by the determination unit 353, the calculation unit 354 calculates a range of setting compensation to be received from the suer U1 because the supporter U2 provides support work that can be set by the supporter U2 (step S103).

Thereafter, the output control unit 355 transmits the range of setting compensation that is calculated by the calculation unit 354 to the supporter terminal device 101 of the supporter U2 (step S104).

Subsequently, the acquisition unit 351 acquires, from the supporter terminal device 101, compensation information on compensation for providing the support work that the supporter U2 selects from the range of setting compensation that is displayed on the supporter terminal device 101 (step S105). In this case, the registration unit 352 stores the compensation information acquired by the acquisition unit 351 in association with the supporter ID in the supporter information storage unit 342.

Thereafter, the acquisition unit 351 acquires the matching condition and the user information that are input by the user U1 using the user terminal device 100 (step S106).

Subsequently, based on the user information, the acquisition unit 351 acquires the point information that is associated with the user U1 that the user U1 has from the point information storage unit 344 (step S107).

Thereafter, based on the point information that the user U1 has and the matching condition that is acquired from the user terminal device 100 of the user U1, the extraction unit 356 extracts the supporters U2 who are within the range of points usable by the user U1 and who meet the requested matching condition from the supporter information storage unit 342 (step S108).

Subsequently, the output control unit 355 transmits the list of the supporters U2 extracted by the extraction unit 356 to the user terminal device 100 of the user U1 (step S109).

Thereafter, the acquisition unit 351 acquires selection information on the selection from the list by the user U1 from the user terminal device 100 (step S110).

Subsequently, the output control unit 355 transmits request information requesting provision of the support work from the user U1 to the supporter terminal device 101 of the supporter U2 corresponding to the selection information (step S111).

Thereafter, the determination unit 357 determines whether approval information on approval of provision of the support work is received from the supporter terminal device 101 of the supporter U2 (step S112). When the determination unit 357 determines that the approval information on approval of provision of the support work is received from the supporter terminal device 101 of the supporter U2 (Yes at step S112), the matching server 3 moves to step S113 described below. On the other hand, when the determination unit 357 determines that the approval information on approval of provision of the support work is not received from the supporter terminal device 101 of the supporter U2 (No at step S112), the matching server 3 moves to step S115 described below.

At step S113, the output control unit 355 transmits matching completion information indicating that it is possible to receive provision of the support work from the supporter U2 that is selected by the user U1 to the user terminal device 100 of the user U1.

Subsequently, the management unit 358 makes a payment of the compensation corresponding to the support work from the point information of the user U1 (step S114). Specifically, based on the point information on the user U1 and the point information on the supporter U2 that the point information storage unit 344 stores, the management unit 358 subtracts points corresponding to the setting range of the support work that the supporter U2 provides from the point information on the user U1 and adds the points corresponding to the setting range according to provision of the support work to the point information on the supporter U2, thereby making a payment of compensation from the user U1 to the supporter U2. After step S114, the matching server 3 ends the process.

At step S115, the output control unit 355 is unable to receive provision of the support work from the selected supporter U2 that is selected for the user terminal device 100 and transmits matching failure information indicating that the matching fails. After step S115, the matching server 3 ends the process.

According to the first embodiment described above, when support work that the supporter U2 who teaches the method of using the medical equipment 2 and the medical procedure is provided to the user U1 who requests the support work, because the calculation unit 354 calculates a range of setting compensation that the supporter U2 receives from the user U1 and the output control unit 355 outputs the range of setting that is calculated by the calculation unit 354 to the supporter terminal device 101 that is associated with the supporter U2, it is possible to set the compensation for the support work done by the supporter U2 appropriately and enable matching with the user U1.

According to the first embodiment, because the calculation unit 354 calculates the range of setting compensation that is received from the user U1 because the supporter U2 provides the support work that can be set based on the rate serving as an index representing the quality of the support work that is determined by the determination unit 353, it is possible to appropriately set compensation for the support work done by the supporter U2 and enable matching with the user U1.

According to the first embodiment, because the determination unit 353 determines the rate serving as the index representing the quality of the support work based on the number of times the supporter U2 experiences the medical procedure of the support work that is requested by the user U1, it is possible to determine the rate corresponding to the supporter U2.

### Second Embodiment

A second embodiment will be described next. In the second embodiment, information on a status of a dialogue that a user and a supporter had in the past is acquired and a rate of the supporter is determined based on the acquired dialogue status information. Furthermore, a matching condition for the supporter to match with the user is set. Functional Configuration of Matching Server

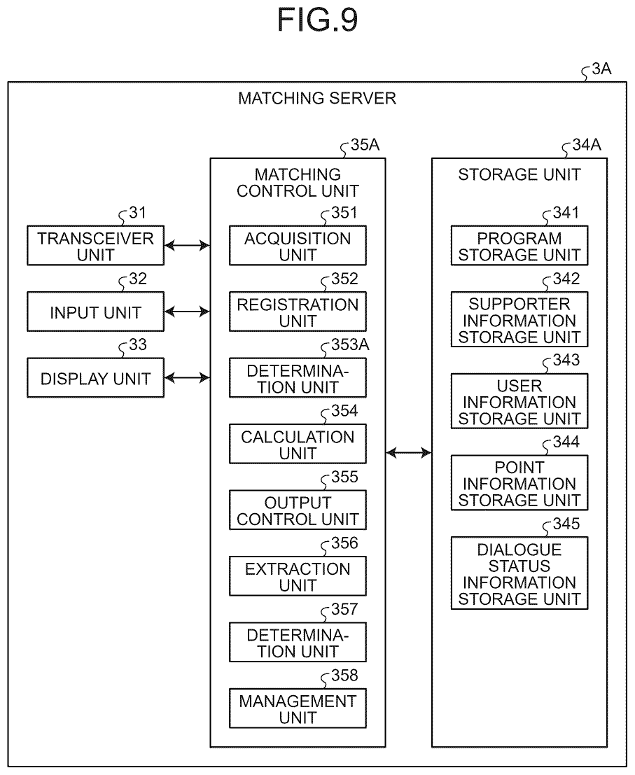

FIG. 9 is a block diagram illustrating a functional configuration of a matching server according to the second embodiment. A matching server 3A illustrated in FIG. 9 includes a storage unit 34A and a matching control unit 35A instead of the storage unit 34 and the matching control unit 35 of the matching server 3 according to the first embodiment described above.

The storage unit 34A includes a dialogue status information storage unit 345 in addition to the configuration of the storage unit 34 according to the above-described first embodiment.

The dialogue status information storage unit 345 stores dialogue status information indicating a dialogue status, such as the content of dialogue with another user, the content of coaching, and the like, at the time when the supporter U2 provided support work in the past. The content of dialogue is, for example, audio data, text data, and keywords important in medical procedure and the method of using the medical equipment 2, etc.

The matching control unit 35A includes a determination unit 353A instead of the determination unit 353 of the matching control unit 35 according to the above-described first embodiment.

The determination unit 353A determines a rate of the supporter U2 based on supporter information that the supporter information storage unit 342 stores and the dialogue statue information that the dialogue status information storage unit 345 stores.

Process performed by Matching Server

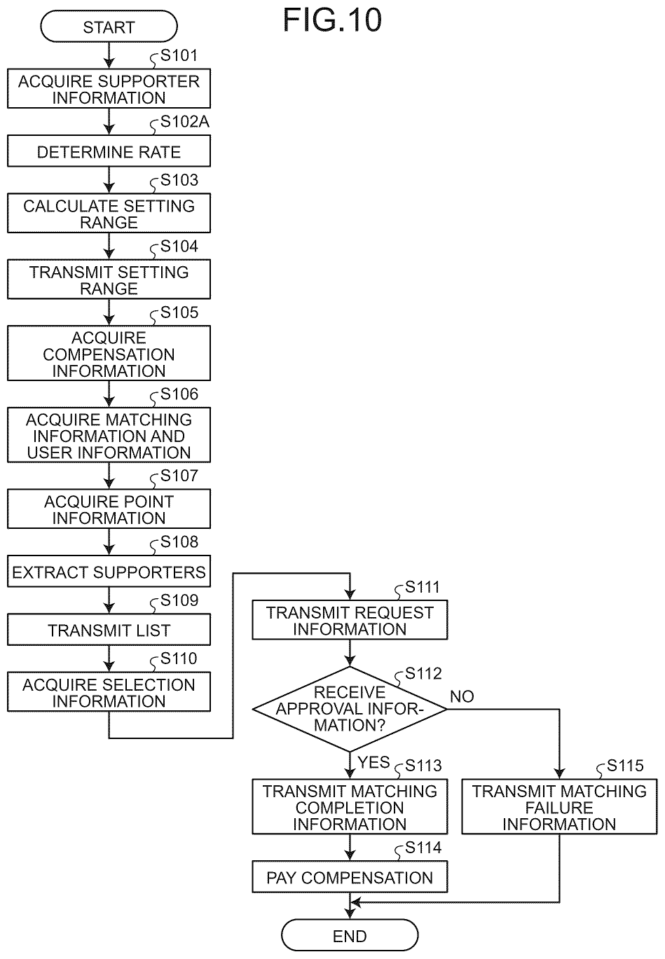

A process that the matching server 3A executes will be described next. FIG. 10 is a flowchart illustrating an overview of the process that the matching server 3A executes. In FIG. 10, the matching server 3A executes step S102A instead of step S102 in FIG. 8 described above and each set of processing excluding step S102A is the same processing as that in FIG. 10 and thus detailed description will be omitted. For this reason, step S102A will be described below.

At step S102A, the determination unit 353A determines a rate of the supporter U2 based on the supporter information that the supporter information storage unit 342 stores and the dialogue status information that the dialogue status information storage unit 345 stores. After step S102A, the matching server 3A moves to step S103.

According to the second embodiment described above, because the determination unit 353A determines a rate of the supporter U2 based on the supporter information stored in the supporter information storage unit 342 and the dialogue status information stored in the dialogue status information storage unit 345, it is possible to determine a rate of the supporter U2 that reflects the content of teaching the medical procedure at the time of provision of the support work to the user U1 and thus it is possible to set compensation for the support work done by the supporter U2 appropriately and enable matching with the user U1.

### Third Embodiment

A third embodiment will be described next. In the third embodiment, a rate of a supporter is determined based on a status of use of medical equipment that the supporter used in the past. Furthermore, an input of a matching condition that specifies a level of proficiency of a user for the supporter to match with the user is received. Functional Configuration of Matching Server

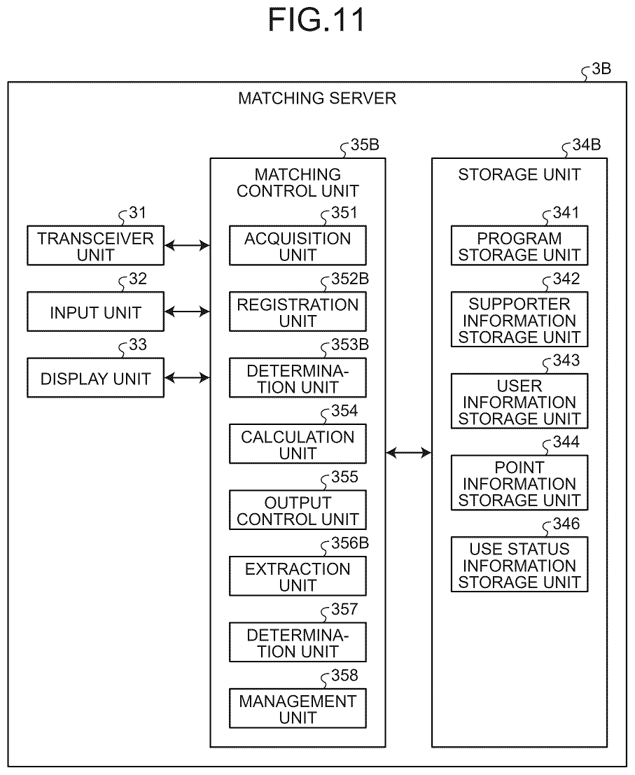

FIG. 11 is a block diagram illustrating a functional configuration of a matching server according to the third embodiment. A matching server 3B illustrated in FIG. 11 includes a storage unit 34B and a matching control unit 35B instead of the storage unit 34 and the matching control unit 35 of the matching server 3 according to the first embodiment described above.

The storage unit 34B includes a use status information storage unit 346 in addition to the configuration of the storage unit 34 according to the above-described first embodiment.

The use status information storage unit 346 stores the supporter ID of the supporter U2 and a status of use of the medical equipment 2 used by the supporter U2 in association with each other. The status of use here is at least one of information on a posture of the medical equipment 2 that is detected by the detection unit 200 of the medical equipment 2, data of images captured by the imaging unit 202, and an input operation in the input unit 203 of the medical equipment 2.

The matching control unit 35B includes a registration unit 352B, a determination unit 353B, and an extraction unit 356B instead of the registration unit 352, the determination unit 353, and the extraction unit 356 of the matching control unit 35 according to the above-described first embodiment.

The registration unit 352 stores the supporter information on the supporter U2 that is acquired by the acquisition unit 351 and the use status information on the medical equipment 2 used by the supporter U2 in association with each other in the use status information storage unit 346.

The determination unit 353B determines a rate of the supporter U2 based on supporter information that the supporter information storage unit 342 stores and the use statue information that the use status information storage unit 346 stores.

Based on matching information that the user U1 specifies and a matching condition on the level of proficiency of the user U1 in medical procedure or the level of proficiency of the user U1 on the medical equipment 2 that the supporter U2 specifies, the extraction unit 356B extracts the supporters U2 who are able to provide the support work requested by the user U1 from the supporter information storage unit 342.

Process performed by Matching Server

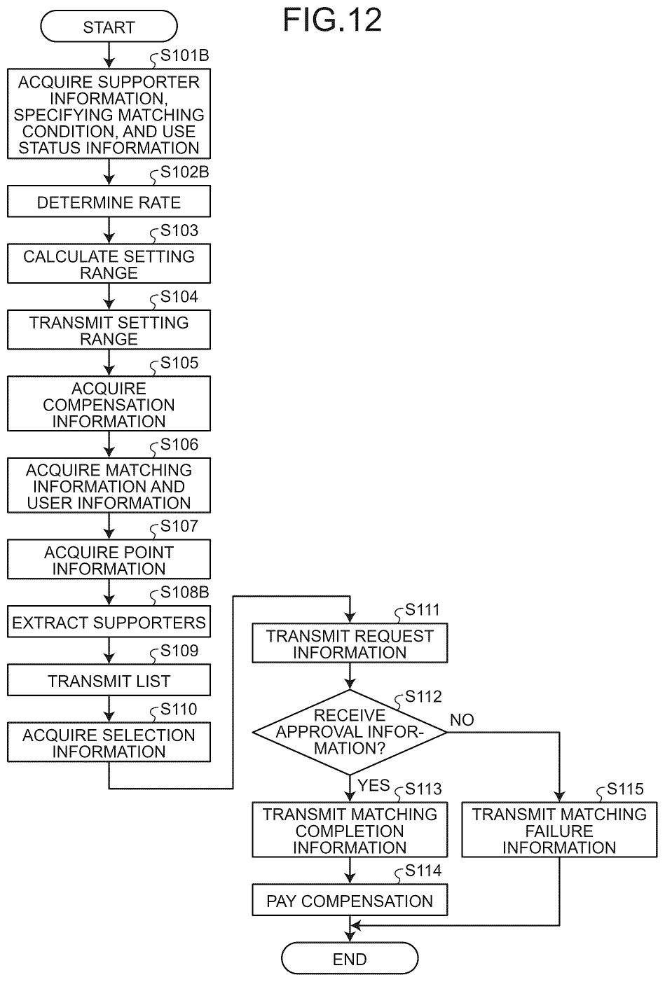

A process that the matching server 3B executes will be described next. FIG. 12 is a flowchart illustrating an overview of the process that the matching server 3B executes. In FIG. 12, instead of step S101, step S102, and step S108, step S101B, step S102B, and step S108B are executed and each set of processing excluding the steps is the same processing as that in FIG. 10 and thus detailed description will be omitted. For this reason, each of step S101B, step S102B, and step S108B will be described below.

At step S101B, the acquisition unit 351 acquires the supporter information on the supporter U2, a specifying matching condition, and use status information that are input by the supporter U2 using the supporter terminal device 101 via the network N100. In this case, the registration unit 352B stores the supporter information that is acquired by the acquisition unit 351 in the supporter information storage unit 342. Furthermore, the registration unit 352B stores the user status information that is acquired by the acquisition unit 351 in the use status information storage unit 346. After step S101B, the matching server 3B moves to step S102B.

At step S102B, the determination unit 353B determines a rate of the supporter U2 based on the supporter information that the supporter information storage unit 342 stores and the use status information that the use status information storage unit 346 stores. After step S102B, the matching server 3B moves to step S103.

At step S108B, based on the matching condition that is specified by the user U1 and the specifying matching condition on the level of proficiency of the user U1 in medical procedure or the level of proficiency of the user U1 on the medical equipment 2 that the supporter U2 specifies, the extraction unit 356B extracts the supporters U2 who are able to provide the support work requested by the user U1 from the supporter information storage unit 342. After step S108B, the matching server 3B moves to step S109.

According to the third embodiment described above, because the determination unit 353B determines a rate of the supporter U2 based on the supporter information stored in the supporter information storage unit 342 and the use status information stored in the use status information storage unit 346 and thus determines a rate reflecting the status of actual use of the medical equipment 2 by the supporter U2, it is possible to set compensation for the support work done by the supporter U2 appropriately and enable matching with the user U1.

According to the third embodiment, because the extraction unit 356B extracts the supporters U2 who are able to provide the support work that the user U1 requests from the supporter information storage unit 342 based on the matching condition that is specified by the user U1 and the specifying matching condition on the level of proficiency of the user U1 in medical procedure or the level of proficiency of the user U1 on the medical equipment 2 that is specified by the supporter U2, the level of proficiency of the user U1 in medical procedure is also taken into consideration and accordingly matching is enabled with fairness between the user U1 and the supporter U2 being ensured.

### Fourth Embodiment

A fourth embodiment will be described next. In the fourth embodiment, a rate of a supporter is determined according to a result of comparing a level of proficiency of a user in a case in the past and a level of proficiency of the user in a case for which support work is received from the supporter.

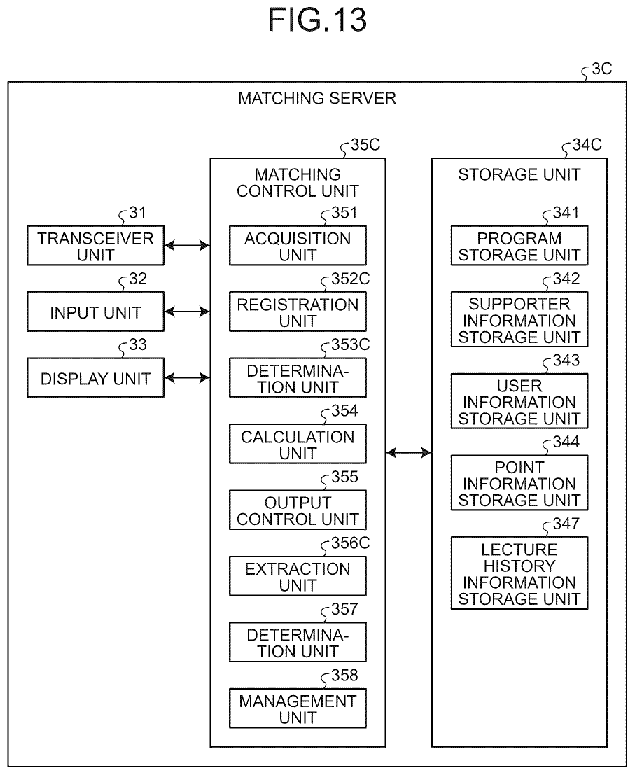

Functional Configuration of Matching Server FIG. 13 is a block diagram illustrating a functional configuration of a matching server according to the fourth embodiment. A matching server 3C illustrated in FIG. 13 includes a storage unit 34C and a matching control unit 35C instead of the storage unit 34 and the matching control unit 35 of the matching server 3 according to the first embodiment.

The storage unit 34C further includes a lecture history information storage unit 347 in addition to the configuration of the storage unit 34 according to the above-described first embodiment.

The lecture history information storage unit 347 stores proficiency level information on a degree of improvement based on the result of comparing a level of proficiency of the user in medical procedure in a case in the past and a level of proficiency of the user in medical procedure in a case for which the user receives support work from the supporter in association with each user ID and each supporter ID.

The matching control unit 35C includes a registration unit 352C, a determination unit 353C, and an extraction unit 356C instead of the registration unit 352, the determination unit 353, and the extraction unit 356 of the matching control unit 35 according to the above-described first embodiment.

The registration unit 352C registers, in the lecture history information storage unit 347, the lecture history information that is acquired by the acquisition unit 351 and that is on the degree of improvement based on the result of comparing a level of proficiency in medical procedure that the user U1 did in the past and a level of proficiency in medical procedure that the user U1 did with reception of provision of support work.

The determination unit 353C determines a rate of the supporter U2 based on the supporter information that is acquired by the acquisition unit 351 and the lecture history information that the lecture history information storage unit 347 stores.

Based on matching information that the user U1 specifies and the matching condition on the level of proficiency of the user U1 in medical procedure or the level of proficiency on the medical equipment 2 that the supporter U2 specifies, the extraction unit 356C extracts the supporters U2 who are able to provide support work that the user U1 requests from the supporter information storage unit 342.

Process performed by Matching Server

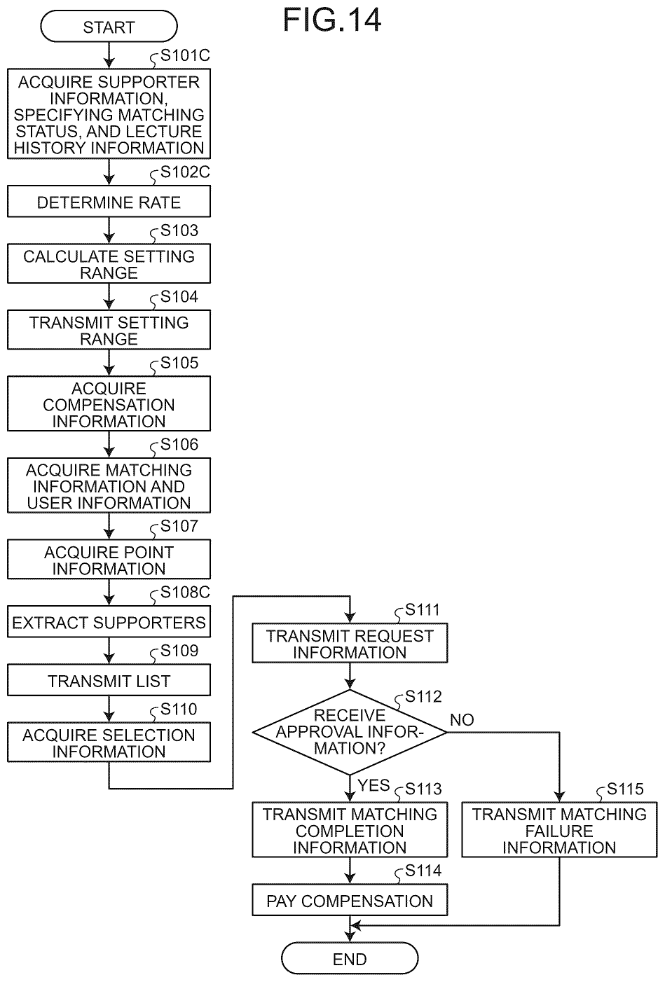

A process that the matching server 3C executes will be described next. FIG. 14 is a flowchart illustrating an overview of the process that the matching server 3C executes. In FIG. 14, the matching server 3C executes step S101C, step S102C, and step S108C instead of step S101, step S102, and step S108 in FIG. 8 described above.

At step S101C, the acquisition unit 351 acquires the supporter information on the supporter U2, a specifying matching condition, and lecture history information that are input by the supporter U2 using the supporter terminal device 101 via the network N100. In this case, the registration unit 352C stores the supporter information that is acquired by the acquisition unit 351 in the supporter information storage unit 342, thereby registering the supporter information. Furthermore, the registration unit 352C stores the lecture history information that is acquired by the acquisition unit 351 in the lecture history information storage unit 347. After step S101B, the matching server 3B moves to step S102C.

At step S102C, the determination unit 353C determines a rate of the supporter U2 based on the supporter information that is acquired by the acquisition unit 351 and the lecture history information that the lecture history information storage unit 347 stores. Specifically, based on the proficiency level information that is associated with the supporter ID of the supporter U2, the determination unit 353C determines a rate such that, the higher the degree of improvement based on the result of comparing the level of proficiency of the user in medical procedure in a case in the past and a level of proficiency of the user in medical procedure in a case for which the user U1 receives support work from the supporter is, the higher the rate is.

At step S108C, based on the matching information that the user U1 specifies and the matching condition on the level of proficiency of the user U1 in medical procedure or the level of proficiency on the medical equipment 2 that the supporter U2 specifies, the extraction unit 356C extracts the supporters U2 who are able to provide the support work that the user U1 requests from the supporter information storage unit 342. After step S108C, the matching server 3C moves to step S109.

According to the fourth embodiment described above, because the determination unit 353C determines a rate of the supporter U2 based on the supporter information stored acquired by the acquisition unit 351 and the lecture history information that is stored in the lecture history information storage unit 347 and thus determines a rate reflecting the improvement in the level of proficiency of the user U1 because of the support work that is actually provided by the supporter U2, it is possible to set compensation for the support work done by the supporter U2 appropriately and enable matching with the user U1.

### Other Embodiments

In the matching system according to the first to fourth embodiments of the disclosure, a single user pays points of the single user as compensation to a supporter when the user receives provision of support work from the supporter; however, the provision is not limited to this and, for example, two or more users may receive provision of support work at the same time, or the like, from the same supporter. In this case, the points the two or more users who receive the support work have may be combined or divided and may be paid as the compensation to the supporter.

In the matching system according to the first to fourth embodiments of the disclosure, the user purchases points from a given point issuing server using a credit card; however the points are not limited to this and, for example, the user may be given with points by browsing a given advertisement or answering a questionnaire.

In the matching system according to the first to fourth embodiments of the disclosure, the matching server performs processing, such as the above-described various types of calculation and output; however, the processing is not limited to this and, for example, part of the processing may be performed in a distributed manner using medical equipment and a terminal device that a supporter owns and medical equipment or a user terminal device that the user owns. For example, a terminal device that a user or a supporter owns may perform processing of calculating a range of setting compensation to be received from the user because the supporter provides support work that can be set, and the like.

In the matching system according to the first to fourth embodiments of the disclosure, the matching server is an independent server; however, it is not limited to this and the matching server may be realized in a way that part of the function of the matching server is separated to another server. Specifically, in the matching system according to the first to fourth embodiments of the disclosure, an independent server may be provided for each function module of the matching server.

It is possible to form a variety of embodiments by appropriately combining a plurality of the components disclosed in the matching system according to the first to fourth embodiments of the disclosure. For example, some components may be deleted from all the components described in the matching system according to the embodiments of the disclosure described above. Furthermore, the components described in the matching system according to the embodiments of the disclosure described above may be combined appropriately.

In the matching system according to the first to fourth embodiments of the disclosure, the “unit” described above may be read as “means”, “circuitry”, or the like. For example, the control unit may be read as a control means or a control circuitry.

The programs that the matching system according to the first to fourth embodiments of the disclosure is caused to execute are recorded in a computer-readable recording medium, such as a CD-ROM, a flexible disc (FD), a CD-R, a DVD (Digital Versatile Disk), a USB medium, or a flash memory, in file data in an installable or executable form and are provided.

The programs that the matching system according to the first to fourth embodiments of the disclosure is caused to execute may be stored in a computer that is connected to a network, such as the Internet, and may be configured such that the programs are downloaded via the network and thus are provided.

According to the disclosure, an effect that it is possible to set compensation for support work done by a supporter appropriately is realized.

In the description of the flowcharts herein, the context of the process among steps is clearly specified using expressions, such as “first of all”, “thereafter”, and “subsequently”; however, the order of the processes necessary to implement the embodiments is not uniquely determined by those expressions. In other words, the order of processes in the flowcharts described herein is changeable within a range without inconsistency.

Some embodiments of the present application have been described above in detail according to the drawings and the embodiments are exemplary and, starting from the modes described in the disclosure section, the present disclosure can be carried out in other modes on which various modifications and improvements are made according to the knowledge of those skilled in the art.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

### Examples

[1] A matching server comprising a processor configured to:
   in matching a user who requests support work of teaching a method of using medical equipment and manipulation and a supporter who provides the support work to the user to receive compensation from the user, calculate a setting range representing a range of the compensation that the supporter is able to set; and notify the supporter of the setting range. [2] The matching server according to [1], wherein the processor is configured to determine the compensation for the support work when the supporter sets a requested value in the setting range.
[3] The matching server according to [1], wherein the processor is configured to
   determine a rate serving as an index representing quality of the support work based on supporter information on the supporter, and calculate the setting range based on the rate.
[4] The matching server according to [3], wherein the supporter information
   contains the number of times of experience of the supporter for each set of manipulation, and
   contains the number of times of experience of the supporter in manipulation for which the user requests the support work, and
   the processor is configured to
   acquire the supporter information, and
   determine the rate based on the number of times of experience of the supporter in the manipulation of the support work that the user requests.
[5] The matching server according to [3], wherein the processor is configured to
   acquire the supporter information and a dialogue status information representing a status of a dialogue at a time of provision of the support work by the supporter in the past, and
   determine the rate based on the supporter information and the dialogue status information.
[6] The matching server according to [3], wherein the processor is configured to
   acquire the supporter information and use status information representing a status of use of medical equipment used in manipulation by the supporter, and
   determine the rate based on the supporter information and the use situation information.
[7] The matching server according to [3], wherein the processor is configured to
   acquire the supporter information and lecture history information on a degree of improvement, the lecture history information on the degree of improvement being based on a result of comparing a level of proficiency in manipulation that the user did in the past with a level of proficiency in manipulation that the user did with reception of provision of support work, and
   determine the rate based on the supporter information and the lecture history information.
[8] The matching server according to [3], wherein the processor is configured to
   acquire user information containing at least a level of proficiency of manipulation that the user did in the past and the supporter information, and
   calculate the setting range based on the user information and the rate.
[9] The matching server according to [1], wherein
   the user and the supporter are doctors, and
   the support work is teaching a method of using medical equipment and manipulation.
[10] The matching server according to [1], wherein the processor is configured to
   acquire point information on points that are associated with each of the user and the supporter,
   subtract points according to the setting range from the point information of the user when the user receives provision of the support work from the supporter, and
   add the points according to the setting range to the point information of the supporter.
[11] The matching server according to [10], wherein the processor is configured to
   acquire the point information that is associated with the user,
   refer to a supporter information storage in which supporter information on each of a plurality of supporters and the setting range of each of supporters are associated,
   extract the supporter who is able to provide the support work for manipulation that the user requests within a range of the points usable by the user, and
   output matching completion information indicating that it is possible to receive the support work from the extracted supporter to a user terminal device that is associated with the user.
[12] The matching server according to [11], wherein the processor is configured to
   acquire matching information that specifies the manipulation that the user requests and,
   based on the matching information, extract the supporter who is able to provide the support work for the manipulation that the user requests from the supporter information storage.
[13] The matching server according to [11], wherein the processor is configured to
   acquire matching information that specifies the manipulation that the user requests and specifying matching condition that specifies a level of proficiency in manipulation by the user who receives provision of the support work, and,
   based on the matching information and the specifying matching condition, extract the supporter who is able to provide the support work for the manipulation that the user requests from the supporter information storage.
[14] A matching system comprising:
   a matching server including a processor;
   a supporter terminal device that is associated with a supporter who teaches a method of using medical equipment and manipulation; and
   a user terminal device that is associated with a user who requests support work that the supporter provides,
wherein
   the processor is configured to
      in matching the user who requests the support work of teaching the method of using the medical equipment and the manipulation and the supporter who provides the support work to the user to receive compensation from the user, calculate a setting range representing a range of the compensation that the supporter is able to set, and
   notify the supporter of the setting range.
[15] A matching method executed by a matching server including a processor, the processor is configured to execute,
   in matching a user who requests support work of teaching a method of using medical equipment and manipulation and a supporter who provides the support work to the user to receive compensation from the user, calculating a setting range representing a range of the compensation that the supporter is able to set, and
   notifying the supporter of the setting range.
[16] A matching server comprising a processor configured to:
   in matching a user who requests support work and a supporter who provides the support work to the user to receive compensation from the user, calculate a setting range representing a range of the compensation that the supporter is able to set; and
   notify the supporter of the setting range.
[17] The matching server according to [16], wherein the processor is configured to determine the compensation for the support work when the supporter sets a requested value in the setting range.
[18] The matching server according to [16], wherein the processor is configured to
   determine a rate serving as an index representing quality of the support work based on supporter information on the supporter, and
   calculate the setting range based on the rate.
[19] The matching server according to [18], wherein the supporter information
   contains the number of times of experience of the supporter for each set of manipulation, and
   contains the number of times of experience of the supporter in manipulation for which the user requests the support work, and
   the processor is configured to
   acquire the supporter information, and
   determine the rate based on the number of times of experience of the supporter in the manipulation of the support work that the user requests.
[20] The matching server according to [18], wherein the processor is configured to
   acquire the supporter information and a dialogue status information representing a status of a dialogue at a time of provision of the support work by the supporter in the past, and
   determine the rate based on the supporter information and the dialogue status information.
[21] The matching server according to [18], wherein the processor is configured to
   acquire the supporter information and use status information representing a status of use of medical equipment used in manipulation by the supporter, and
   determine the rate based on the supporter information and the use situation information.
[22] The matching server according to [18], wherein the processor is configured to
   acquire the supporter information and lecture history information on a degree of improvement, the lecture history information on the degree of improvement being based on a result of comparing a level of proficiency in manipulation that the user did in the past with a level of proficiency in manipulation that the user did with reception of provision of support work, and
   determine the rate based on the supporter information and the lecture history information.
[23] The matching server according to [18], wherein the processor is configured to
   acquire user information containing at least a level of proficiency of manipulation that the user did in the past and the supporter information, and
   calculate the setting range based on the user information and the rate.
[24] The matching server according to [16], wherein
   the user and the supporter are doctors, and
   the support work is teaching a method of using medical equipment and manipulation.
[25] The matching server according to [16], wherein the processor is configured to
   acquire point information on points that are associated with each of the user and the supporter,
   subtract points according to the support work from the point information of the user when the user receives provision of the support work from the supporter, and
   add the points according to the support work to the point information of the supporter.
[26] The matching server according to [25], wherein the processor is configured to
   acquire the point information that is associated with the user,
   based on supporter information on each of a plurality of supporters and compensation information on the support work of each of supporters, extract the supporter who is able to provide the support work for manipulation that the user requests within a range of the points usable by the user from the plurality of supporters, and
   notify the user of matching completion information indicating that it is possible to receive the support work from the extracted supporter.
[27] The matching server according to [26], wherein the processor is configured to
   acquire matching information that specifies the manipulation that the user requests and,
   based on the matching information, extract the supporter who is able to provide the support work for the manipulation that the user requests from the plurality of supporters.
[28] The matching server according to [26], wherein the processor is configured to
   acquire matching information that specifies the manipulation that the user requests and specifying matching condition that specifies a level of proficiency in manipulation by the user who receives provision of the support work, and,
   based on the matching information and the specifying matching condition, extract the supporter who is able to provide the support work for the manipulation that the user requests from the plurality of supporters.
[29] The matching server according to [26], further comprising a supporter information storage, wherein
   the processor is configured to
   store the supporter information on each of the plurality of supporters and the compensation information on the support work of each of supporters in association with each other in the supporter information storage, and
   refer to the supporter information storage to extract the supporter who is able to provide the support work for manipulation that the user requests within the range of the points usable by the user.
[30] The matching server according to [29], wherein the processor is configured to
   acquire matching information that specifies the manipulation that the user requests and,
   based on the matching information, extract the supporter who is able to provide the support work for the manipulation that the user requests from the supporter information storage.
[31] The matching server according to [29], wherein the processor is configured to
   acquire matching information that specifies the manipulation that the user requests and specifying matching condition that specifies a level of proficiency in manipulation by the user who receives provision of the support work, and,
   based on the matching information and the specifying matching condition, extract the supporter who is able to provide the support work for the manipulation that the user requests from the supporter information storage.
[32] A matching system comprising:
   a matching server including a processor; and
   a supporter terminal device that is associated with a supporter who provides support work to a user to receive compensation from the user, wherein
   the processor is configured to
      in matching the user who requests the support work and the supporter, calculate a setting range representing a range of the compensation that the supporter is able to set, and
      output the setting range to the supporter terminal device.
[33] The matching system according to [32], further comprising a user terminal device that is associated with the user, wherein
   the processor is configured to
   acquire user information from the user terminal device,
   acquire supporter information from the supporter terminal device,
   determine a rate serving as an index representing quality of the support work based on the supporter information, and
   calculate the setting range based on the user information and the rate.
[34] The matching system according to [32], further comprising a user terminal device that is associated with the user, wherein
   the processor is configured to
   acquire point information that is associated with the user of the user terminal device,
   based on supporter information on each of a plurality of supporters and compensation information on the support work of each of supporters, extract a supporter candidate who is able to provide the support work for manipulation that the user requests within a range of the points usable by the user from the plurality of supporters,
   transmit a list of the extracted supporter candidate to the user terminal device,
   transmit request information requesting provision of the support work from the user to the supporter terminal device when selection information on the supporter selected from the list is acquired from the user terminal device,
   determine whether approval information on approval of provision of the support work is received from the supporter terminal device, and
   submit a determination result of reception of the approval information.
[35] A matching method executed by a matching server including a processor, the processor is configured to execute,
   in matching a user who requests support work and a supporter who provides the support work to the user to receive compensation from the user, calculating a setting range representing a range of the compensation that the supporter is able to set, and
   notifying the supporter of the setting range.

What is claimed is:

1. A matching server comprising a processor configured to:
in matching a user who requests support work and a supporter who provides the support work to the user to receive compensation from the user, calculate a setting range of the compensation that the supporter is able to set; and
notify the supporter of the setting range.

2. The matching server according to claim 1, wherein the processor is configured to determine the compensation for the support work when the supporter sets a requested value in the setting range.

3. The matching server according to claim 1, wherein the processor is configured to
determine a rate serving as an index representing quality of the support work based on supporter information on the supporter, and
calculate the setting range based on the rate.

4. The matching server according to claim 3, wherein the supporter information comprises:
a number of times of experience of the supporter for each set of medical procedure, and
a number of times of experience of the supporter in medical procedure for which the user requests the support work, and
the processor is configured to:
acquire the supporter information, and
determine the rate based on the number of times of experience of the supporter in the medical procedure of the support work that the user requests.

5. The matching server according to claim 3, wherein the processor is configured to:
acquire the supporter information and a dialogue status information representing a status of a dialogue at a time of provision of the support work by the supporter in the past, and
determine the rate based on the supporter information and the dialogue status information.

6. The matching server according to claim 3, wherein the processor is configured to
acquire the supporter information and use status information representing a status of use of medical equipment used in medical procedure by the supporter, and
determine the rate based on the supporter information and the use status information.

7. The matching server according to claim 3, wherein the processor is configured to
acquire the supporter information and lecture history information representing a degree of improvement, the lecture history information being based on a result of comparing a level of proficiency in medical procedure that the user did in the past with a level of proficiency in medical procedure that the user did with reception of provision of support work, and
determine the rate based on the supporter information and the lecture history information.

8. The matching server according to claim 3, wherein the processor is configured to:
acquire user information comprising at least a level of proficiency of medical procedure that the user did in the past and the supporter information, and
calculate the setting range based on the user information and the rate.

9. The matching server according to claim 1, wherein
the user and the supporter are doctors, and
the support work is teaching a method of using medical equipment and medical procedure.

10. The matching server according to claim 1, wherein the processor is configured to:
acquire point information on points that are associated with each of the user and the supporter,
subtract points according to the support work from the point information of the user when the user receives provision of the support work from the supporter, and
add the points according to the support work to the point information of the supporter.

11. The matching server according to claim 10, wherein the processor is configured to:
acquire the point information that is associated with the user,
based on supporter information each associating a plurality of supporters with compensation information representing the support work of each of supporters, extract the supporter who is able to provide the support work for medical procedure that the user requests within a range of the points usable by the user from the plurality of supporters, and
notify the user of matching completion information indicating that it is possible to receive the support work from the extracted supporter.

12. The matching server according to claim 11, wherein the processor is configured to
acquire matching information that specifies the medical procedure that the user requests and,
based on the matching information, extract the supporter who is able to provide the support work for the medical procedure that the user requests from the plurality of supporters.

13. The matching server according to claim 11, wherein the processor is configured to
acquire matching information that specifies the medical procedure that the user requests and specifying matching condition that specifies a level of proficiency in medical procedure by the user who receives provision of the support work, and,
based on the matching information and the specifying matching condition, extract the supporter who is able to provide the support work for the medical procedure that the user requests from the plurality of supporters.

14. The matching server according to claim 11, further comprising a supporter information storage, wherein
the processor is configured to
store the supporter information on each of the plurality of supporters and the compensation information on the support work of each of supporters in association with each other in the supporter information storage, and
refer to the supporter information storage to extract the supporter who is able to provide the support work for medical procedure that the user requests within the range of the points usable by the user.

15. The matching server according to claim 14, wherein the processor is configured to:
acquire matching information that specifies the medical procedure that the user requests and,
based on the matching information, extract the supporter who is able to provide the support work for the medical procedure that the user requests from the supporter information storage.

16. The matching server according to claim 14, wherein the processor is configured to
acquire matching information that specifies the medical procedure that the user requests and specifying matching condition that specifies a level of proficiency in medical procedure by the user who receives provision of the support work, and,
based on the matching information and the specifying matching condition, extract the supporter who is able to provide the support work for the medical procedure that the user requests from the supporter information storage.

17. A matching system comprising:
a matching server including a processor; and
a supporter terminal device that is associated with a supporter who provides support work to a user to receive compensation from the user, wherein
the processor is configured to
in matching the user who requests the support work and the supporter, calculate a setting range of the compensation that the supporter is able to set, and
output the setting range to the supporter terminal device.

18. The matching system according to claim 17, further comprising a user terminal device that is associated with the user, wherein
the processor is configured to:
acquire user information from the user terminal device,
acquire supporter information from the supporter terminal device,
determine a rate serving as an index representing quality of the support work based on the supporter information, and
calculate the setting range based on the user information and the rate.

19. The matching system according to claim 17, further comprising a user terminal device that is associated with the user, wherein
the processor is configured to:
acquire point information that is associated with the user of the user terminal device,
based on supporter information each associating a plurality of supporters with compensation information representing the support work of each of supporters, extract a supporter candidate who is able to provide the support work for medical procedure that the user requests within a range of the points usable by the user from the plurality of supporters,
transmit a list of the extracted supporter candidate to the user terminal device,
transmit request information requesting provision of the support work from the user to the supporter terminal device when selection information on the supporter selected from the list is acquired from the user terminal device,
determine whether approval information on approval of provision of the support work is received from the supporter terminal device, and
submit a determination result of reception of the approval information.

20. A matching method executed by a matching server including a processor, the processor is configured to execute,
in matching a user who requests support work and a supporter who provides the support work to the user to receive compensation from the user, calculating a setting range of the compensation that the supporter is able to set, and
notifying the supporter of the setting range.
